(19) United States
(12) Patent Application Publication
(10) Pub. No.: US 2007/0293860 A1
(43) Pub. Date: Dec. 20, 2007

(54) Elastic Element Produced From Radiolucent Material For A Medical Device

(76) Inventor: Marc Oesch, Bibeiist (CH)

Correspondence Address: JONES DAY, 222 EAST 41ST STREET, NEW YORK, NY, 10017-6702, US

(21) Appl. No.: 11/578,496

(22) PCT Filed: Apr. 19, 2004
(86) PCT No.: PCT/CH04/00237

§ 371 Date: Aug. 30, 2007

(51) Int. Cl. A61B 17/56

(52) U.S. Cl. 606/60; 606/76

(57) ABSTRACT

An elastic element which consists of a radiolucent material and is provided for a medical device. Said elastic element is produced from an unreinforced plastic material.

### FIELD OF THE INVENTION

The invention relates to an elastic element, produced from a radiolucent material for a medical device.

### BACKGROUND OF THE INVENTION

There is a large number of devices in medical technology, such as an external fixator, which must be brought one or more times into the beam path of a source of x-rays for the purpose of checking a position. Since such devices generally consist of metallic materials, which cast a shadow in an x-ray image, structures lying behind are hidden. This is a disadvantage. The proposal has therefore already been made to produce individual parts of such devices from radiolucent materials. However, such a replacement never extends to any elastic elements that may be present, such as springs, which continue to be made from metal. However, such springs affect the quality of x-ray photographs.

### SUMMARY OF THE INVENTION

It is an object of the invention to provide an elastic element, which is made from a radiolucent plastic and is in a position to fulfill all the mechanical functions of a metallic element, however, without casting a shadow on the x-ray image.

The advantages, achieved by the invention, are essentially:
better transmittance of x-rays,
less weight
greater flexibility in comparison to elastic elements from reinforced plastic, which break easily.

The radiolucent plastic may consist of polyoxymethylene (POM). Advisably, the plastic has a modulus of elasticity of 2600-3500 MPa and preferably of 2800 to 3300 MPa. The yield strain of the plastic advisably ranges from 20 to 24% and preferably from 21 to 23%. The nominal elongation at break of the plastic advisably ranges from 40 to 50% and preferably from 43% to 47%.

For a special embodiment, the inventive element has the shape of a helical spring. The cross section of the spring spiral of the helical spring advantageously is out-of-round and preferably is quadrilateral. Advantageously, the cross section of the spring spiral tapers towards the outside of the helical spring, preferably in a trapezoidal manner.

For a different embodiment, the inventive element is constructed as a disk spring. The disk spring preferably is configured in the shape of a hollow, truncated cone and tapers from the first to the second end (31; 32) and has several notches, which are distributed over the periphery and indented from the first end. The notches increase the elasticity of the disk spring and prevent breakage of the same. The conical angle of the disk spring in the shape of a hollow truncated cone may advantageously be between 70° and 140°, which leads to an optimum overall height.

The invention relates to a medical device, all the components of which, with the exception of the elastic element, consist of a reinforced plastic. Such a device may be realized in the form of a clamp for fastening bone fixation means and/or longitudinal components. The elastic element permits bone fixation means and/or longitudinal components, introduced into the clamp, to be fastened temporarily. All components of the clamp, with the exception of the elastic element, consist of a fiber-reinforced plastic, which may, for example, be a polyamide.

The invention furthermore relates to a bone fixation device for the mutual positioning of the longitudinal components, which comprises two medical devices with an inventive elastic element, which may be rotated with respect to one another about an axis of rotation. Each of the two clamps comprises at least two clamp jaws, which define a clamp opening located between then, it being possible to constrict or expand the clamp opening by the elastic deformation of the clamp. At least one jaw of each clamp has a borehole, which is coaxial with the axis of rotation and traverses the clamp jaw. The two clamps are mounted on a shaft and blocking means are provided in order to press the jaws of the two clamps coaxially against one another, in order alternatively to block the rotatability of the two clamps about the axis of rotation relative to one another.

For a special embodiment, an axially inner clamp jaw is connected permanently with the shaft. The surfaces of the axially inner clamp jaw, directed against one another, may be provided with serrations, which can be brought into engagement with one another. There may also be a disk spring between the axially inner clamp jaws. The blocking means advisably are nuts, which can be screwed axially terminally onto at each end of the shaft and can be pressed against the axially outside clamp jaw. A helical spring may also be disposed between each nut and the adjacent outside clamp jaw.

In the case of a further embodiment, a device to prevent twisting is provided between each axially outside and the adjacent axially inside clamp jaw. Advantageously, caulking material, which preferably is produced by thermal conversion, may be provided appropriately at one end of the shaft.

### BRIEF DESCRIPTION OF THE DRAWINGS

The invention and further developments of the invention are explained in even greater detail in the following by means of partially diagrammatic representations of several examples. In the drawing

FIG. 1 shows a side view of an embodiment of the elastic element, constructed as a helical spring,

FIG. 2 shows a cross-section along the line II-II in FIG. 1,

FIG. 3 shows a perspective view of an embodiment of the elastic element constructed as a disk spring,

FIG. 4 shows a diametrical section through the disk spring shown in FIG. 3,

FIG. 5 shows a perspective view of an embodiment of the bone fixation device,

FIG. 6 shows a side view of the embodiment of the bone fixation device, shown in FIG. 5,

FIG. 7 shows a cross-section along the line III-III in FIG. 6 and

FIG. 8 shows an enlargement of the section marked by the circle A in FIG. 7.

### DETAILED DESCRIPTION OF THE PREFERRED EMBODIMENTS

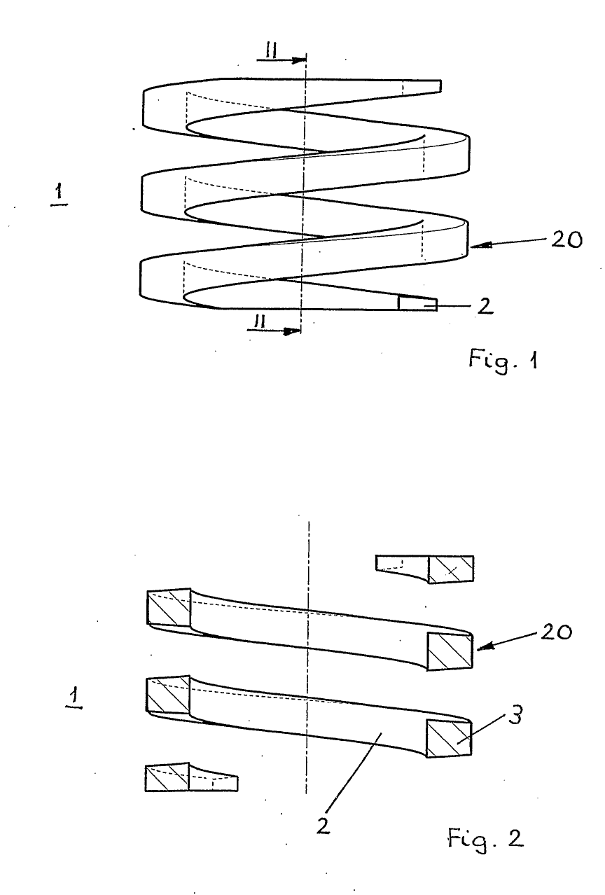

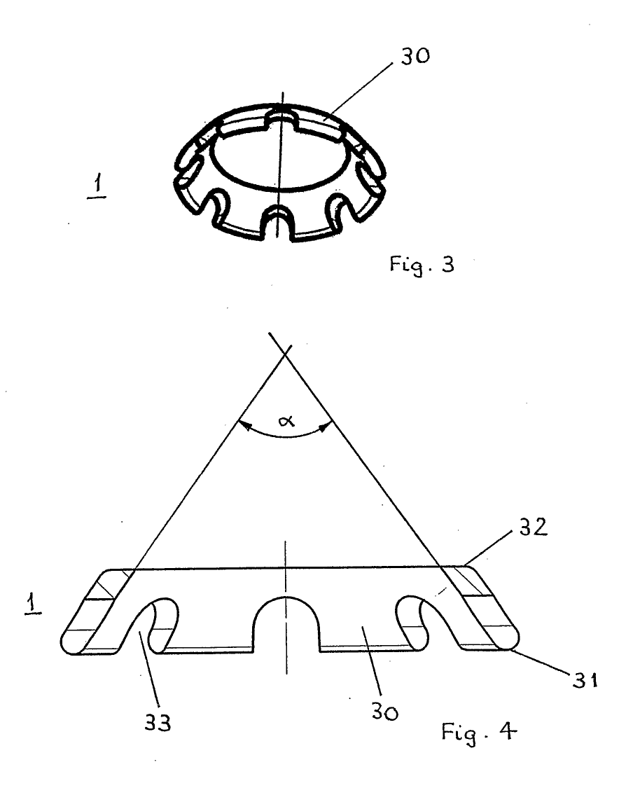

FIGS. 1 and 2 show an embodiment of the elastic element 1, which has the shape of a helical spring 20 with flattened ends. The cross section 3 of the spring spiral 2 is trapezoidal and tapers towards the periphery of the helical spring 20. A further embodiment of the elastic element is shown in FIGS. 3 and 4. The elastic element 1 is constructed as a disk spring 30 and has the configuration of a hollow truncated cone. The disk spring 30 tapers from its first end 31 to its second end 32 and has several notches 33, which are distributed over the periphery and indented from the first and 31. The conical angle α of the disk spring 30 in the shape of a hollow truncated cone is 70° in the embodiment shown here.

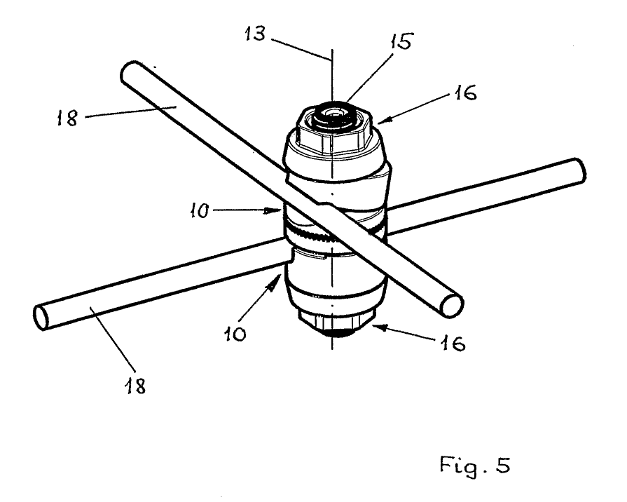

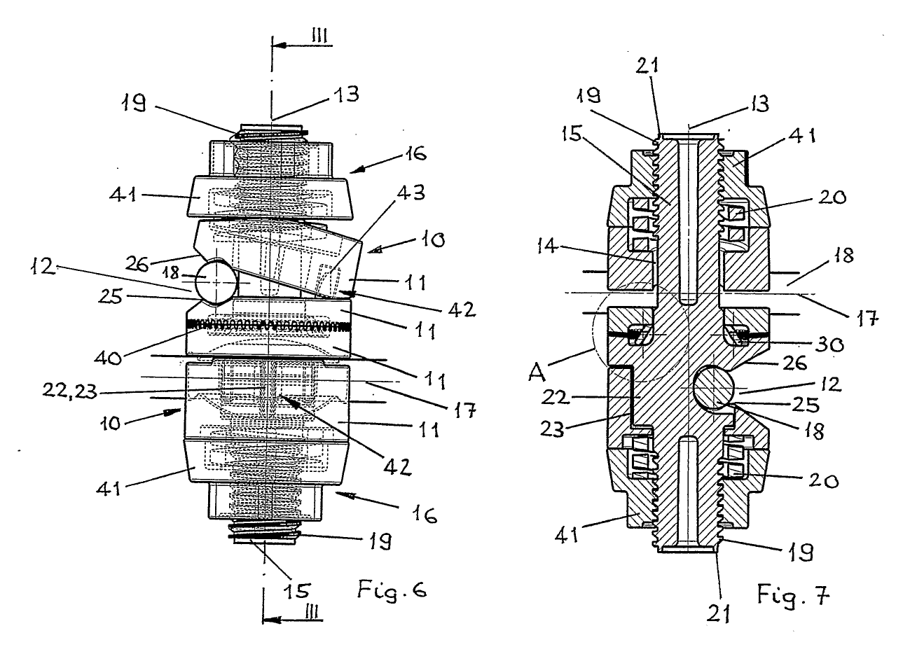

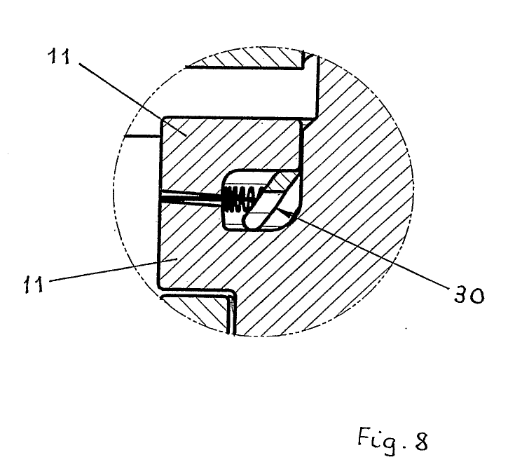

FIGS. 5 to 8 show an embodiment of the bone fixation device, which comprises two clamps 10, which can be rotated about an axis of rotation 13 relative to one another. Each of the clamps 10 comprises two clamp jaws 11, which are disposed one behind the other axially, a clamp opening 12 being disposed between the two jaws 11 of each clamp 10. The clamp openings 12 are configured in the form of a channel and have a channel axis 17 each, which is orthogonal to the axis of rotation 13. The clamp openings 12 are intended to accommodate longitudinal fastening elements 18. The clamp openings 12 are open transversely to the channel axis 17 towards the periphery of the clamp 10, so that a longitudinal fastening elements 18 can be introduced into the clamp opening 12 transversely to the axis of rotation 13.

A borehole 14 passes coaxially with the axis of rotation 13 through the axially outer jaw 11 of the above-disposed clamp 10 as well as the two jaws belonging to the clamp 10 disposed at the bottom. On the other hand, the inner jaw 11 of the clamp 10 disposed at the top is connected permanently with the shaft 15 passing through the borehole 14. The jaws 11 of the clamp 10 disposed at the top, which can be moved rotationally and axially, enable the clamp 10, which is disposed at the top, to be moved rotationally relative to the clamp 10 disposed at the bottom. At the two ends of the shaft 15, external threads 10 are provided, over which the blocking agents 16, configured as nuts 41, can be screwed. The diameters of the borehole 14 and of the shaft 15 are such that the shaft 15 has clearance in the borehole 14.

The nuts 41 are disposed axially terminally at the shaft 15 and press axially on the respectively adjacent axially outer clamp jaw 11, so that, by tightening the upper nut 41, the two jaws 11 of the clamp 10, which is disposed at the top, can be pressed against the inner jaw 11 of the clamp 10, which is disposed at the bottom and fixed to the shaft 15. With that, the two jaws 11 of the clamp 10 can also be pressed against one another. By tightening the nut 41, which is disposed at the bottom, the axially outer clamp jaw 11 of the nut 41, which is disposed at the bottom, is pressed against the clamp jaw 11, which is connected permanently with the shaft 15 and is part of this clamp 10. By these means, the longitudinal fastening elements 18, which are inserted coaxially with the channel axes 17, can be fixed between the clamp jaws 11. Moreover, the mutually adjacent inner face surfaces of the axially inner clamp jaws 11 are provided with serrations 40, which, when the upper nut 41 is tightened, engage one another and prevent relative rotation of the two clamps 10 about the axis of rotation 13.

Each of the clamp openings 12 can be expanded or constricted by elastic deformation of the helical springs 20, disposed axially between the nuts 41 and the outer clamp jaws 11. Furthermore, the axially inner clamp jaw 11 of the clamp 11 disposed at the top is pressed by means of a disk spring 30, inserted between the two axially inner clamp jaws 11, against the axially outer clamp jaw 11 of the clamp 10, which is disposed at the top. By these means, the serrations 40 are disengaged and relative rotation of the two clamps 10 about the axis of rotation 13 is made possible.

The elastic pre-stressing, attainable by the helical springs 20, is achieved by constructing the helical spring 20 appropriately. Moreover, two clamp openings 12 are constructed transversely to the channel axes 17 in such a manner, that they have a constriction 25 at each of their openings, which are remote from the axis of rotation 13. The clamp openings 12 are provided peripherally with expansions 26 to simplify the introduction of a longitudinal fastening element 18 into the clamp opening 12 in question.

Rotation of the two jaws 11 of each clamp 10 about the axis of rotation 13 relative to one another is prevented by a device 42. In the case of the clamp 10, which is disposed at the top, the device 42 is realized by a pin 43, which is disposed between the clamp jaws 11. In the case of the clamp 10, which is disposed at the bottom, the device 42 is realized by key seat 22 at the shaft 15 and a corresponding groove 23.

1-22. (canceled)

23. A medical device comprising:
a clamp comprised of a reinforced radiolucent material; and
an elastic element comprised of a non-reinforced radiolucent material and disposed in the clamp;
wherein the clamp is configured to releasably engage a longitudinal component.

24. The medical device of claim 23, wherein the non-reinforced radiolucent material is plastic.

25. The medical device of claim 24, wherein the plastic has a modulus of elasticity between about 2500 MPa to about 3500 MPa.

26. The medical device of claim 23, wherein the elastic element is a helical spring.

27. The medical device of claim 23, wherein the elastic element is a disc spring.

28. The medical device of claim 23, wherein the reinforced radiolucent material is polyamide.

29. A bone fixation system comprising:
a first clamp having a first engaging surface, having a first elastic element disposed therein, wherein the first clamp is releasably engaged to a first longitudinal component; and
a second clamp having a second engaging surface, having a second elastic element disposed therein, wherein the second clamp is releasably engaged to a second longitudinal component;
wherein the first engaging surface engages the second engaging surface such that the first and second clamps are axially aligned;
wherein the first and second clamps are comprised of a reinforced radiolucent material; and
wherein the first and second elastic elements are comprised of a non-reinforced radiolucent material.

30. The bone fixation system of claim 29, wherein the first longitudinal component extends in a different direction than the second longitudinal component.

31. The bone fixation system of claim 29, wherein the first clamp has at least two jaws for receiving the first longitudinal component therebetween.

32. The bone fixation system of claim 29, wherein the first and second clamps are mounted on a shaft.

33. The bone fixation system of claim 29, further comprising a nut for preventing rotation of the first clamp relative to the second clamp.

34. The bone fixation system of claim 29, wherein the first and second elastic elements are helical springs.

35. The bone fixation system of claim 29, further comprising a third elastic element.

36. The bone fixation system of claim 35, wherein the first and second elastic elements are helical springs, and wherein the third elastic element is a disc spring.

37. The bone fixation system of claim 36, wherein the disc spring is disposed between the helical springs.
